(19) United States
(12) Patent Application Publication
(10) Pub. No.: US 2026/0193581 A1
(43) Pub. Date: Jul. 9, 2026

(54) MOLECULE PRODUCTION SYSTEM AND METHOD

(71) Applicant: COMMONWEALTH SCIENTIFIC AND INDUSTRIAL RESEARCH ORGANISATION, Australian Capital Territory (AU)

(72) Inventor: Oliver Lachlan MEAD, Australian Capital Territory (AU)

(73) Assignee: COMMONWEALTH SCIENTIFIC AND INDUSTRIAL RESEARCH ORGANISATION, Australian Capital Territory (AU)

(21) Appl. No.: 19/133,042

(22) PCT Filed: Nov. 28, 2023
(86) PCT No.: PCT/AU2023/051219

§ 371 Date: May 27, 2025

(30) Foreign Application Priority Data
Nov. 28, 2022 (AU) 2022903607

(51) Int. Cl. C12M 1/00; C12M 1/34

(52) U.S. Cl. CPC C12M 23/58; C12M 29/00; C12M 29/04; C12M 29/18; C12M 41/00

(57) ABSTRACT

A biomass reactor comprising an enclosed fluid network including plural receptacles is disclosed. In an embodiment, each receptacle is for containing a volume of a liquid medium for growing a microbial biomass in or on the liquid medium. The plural receptacles are arranged in fluid communication to allow for a recirculating flow of the liquid medium to cascade through the plural receptacles. A method for producing a molecular product using the disclosed biomass reactor and a product formed by operating the biomass reactor is also disclosed.

### PRIORITY APPLICATION

This international patent application claims priority from Australian Provisional Patent Application No. 2022903607 filed on 28 Nov. 2022, the entire contents of which are herein incorporated by reference.

### TECHNICAL FIELD

The present disclosure relates to biomass processing. In a particular embodiment, the present disclosure relates to biomass processing for producing high-value molecules such as compounds, nucleic acids, lipids and proteins. The present disclosure also relates to biomass processing for detoxifying or reconstituting waste materials.

### BACKGROUND

Technologies exist for processing biomass to produce molecules such as small molecule compounds, nucleic acids, and proteins. These technologies are used for fermentation-based biotechnology and synthetic biology and include “reactors” or “reactor systems” which involve growing a microbial biomass as a conglomerate mat or film on top of a liquid medium in a receptacle or reservoir and extracting molecular by-products from the liquid medium. Biomass is also able to grow as a film or mass along the submerged surface of the containing vessel.

Using existing technology, the microbial biomass may be grown in a number of different ways. For example, it may be grown as fungi in a mat called a ‘syncytium’ where the cellular contents are connected allowing for efficient distribution of nutrients and wastes. In another example, organisms, such as species of bacteria, yeast, and algae, can form a biofilm of individual cells on top of the liquid medium, or cover the submerged surfaces, in a reactor. In other examples, non-biofilm forming cells can be embedded in a solid matrix or gel and floated on top of the liquid medium in a reactor as the biomass.

Traditional reactors comprise a receptacle or reservoir containing a liquid medium which is stirred and aerated. However, since such reactors can contain a large volume of liquid medium, the stirring and aeration equipment can be susceptible to mechanical failures associated with stirring and aerating the large volume. Filamentous organisms can also tend to cause mechanical failures. In addition to presenting a risk of a mechanical failure, stirring and aeration are also expensive and can be a source of contamination resulting in biological failure of the biomass.

Furthermore, growing biomass is resource and time intensive. In some existing reactors where microbial biomass is grown as a conglomerate mat or film on top of the liquid medium, the microbial biomass is disturbed to extract the molecules. In other systems, the liquid medium builds up a toxic concentration of waste products which kills the biomass.

It would be desirable to provide a reactor system which addresses at least some of the above-mentioned deficiencies of existing reactor systems.

### SUMMARY

The following presents a simplified summary of one or more preferred embodiments of the present disclosure, in order to provide a basic understanding of such embodiments. This summary is not an extensive overview of all contemplated embodiments of the disclosure, and is intended neither to identify key or critical elements of all embodiments of the disclosure nor to delineate the scope of any or all embodiments of the disclosure. Its sole purpose is to present some concepts of one or more embodiments of the disclosure in a simplified form as a prelude to the more detailed description that is presented later.

One embodiment of the disclosure provides a biomass reactor, comprising:
an enclosed fluid network including plural receptacles, each receptacle for containing a volume of a liquid medium for growing a microbial biomass in or on the liquid medium,
wherein the plural receptacles are arranged in fluid communication to allow for a recirculating flow of the liquid medium to cascade through the plural receptacles.

In certain embodiments, each receptacle includes plural air inlets, each air inlet being associated with an aperture which is covered or obstructed by a material which permits passive diffusion of air into the internal volume from an external environment via the plural air inlets. In an embodiment, the material is a semipermeable membrane. However, other suitable materials may be used.

In embodiments, the recirculating flow of the liquid medium flows into at least one first receptacle of the plural receptacles via at least one inlet and flows out of at least one final receptacle of the plural receptacles via at least one outlet. The first receptacle and the final receptacle may be in direct fluid communication or they may be in indirect fluid communication involving one or more other receptacles.

Each receptacle of the plural receptacles may include at least one respective inlet and at least one respective outlet. In embodiments, the respective at least one inlet of the at least one first receptacle receives a flow of the liquid medium from a pump of the enclosed fluid network and the respective at least one outlet of the at least one final receptacle drains liquid medium into a sump of the enclosed fluid network. In embodiments, the sump is in fluid communication with the pump so that the pump can pump liquid medium from the sump into the first receptacle.

In certain embodiments, the at least one inlet and the at least one outlet of each respective receptacle is juxtaposed to a base of each respective receptable.

In certain embodiments, the plural receptacles are arranged as a multistage network such that each stage comprises at least one receptacle.

In embodiments, a layer of biomass is disposed on a surface of the liquid medium contained in each receptacle of the enclosed fluid network. For each receptacle, the respective inlet and outlet may be configured to reduce disturbance of the biomass layer during the recirculating flow of the liquid medium.

In certain embodiments, the plural receptacles are arranged as a vertical stack.

Yet another aspect of an embodiment of the disclosure provides a biomass reactor, comprising:
an enclosed fluid network including plural receptacles, each receptacle containing a volume of a liquid medium and a microbial biomass growing in or on the liquid medium, each of the plural receptacles arranged in fluid communication to allow for a recirculating flow of the liquid medium to cascade through the plural receptacles such that the recirculating flow of the liquid medium flows into at least one first receptacle of the plural receptacles via at least one inlet and flows out of at least one final receptacle of the plural receptacles via at least one outlet;
a sump containing an additional volume of the liquid medium, the sump having a sump inlet in fluid communication with the at least one outlet of the enclosed fluid network, and a sump outlet; and
a pump for generating a flow of the liquid medium between the sump outlet and the inlet of the enclosed fluid network to establish and/or maintain the recirculating flow of the liquid medium.

Still another aspect of the present disclosure provides a method for producing a molecular product, comprising:
providing an enclosed fluid network comprising plural vertically offset receptacles arranged in fluid communication, each receptacle containing a volume of a liquid medium for growing a microbial biomass in or on the liquid medium;
establishing fluid communication of the liquid medium between at least one final receptacle of the enclosed fluid network and at least one first receptacle of the enclosed fluid network so as to provide a recirculating flow of the liquid medium through the plural receptacles of the enclosed fluid network; and
processing liquid medium obtained from the at least one final receptacle to extract one or more molecular products from the liquid medium.

Yet another aspect of an embodiment of the disclosure provides a method of forming a biomass reactor for producing a molecular product, comprising:
providing an enclosed fluid network including plural receptacles, each receptacle containing a volume of a liquid medium and a microbial biomass growing in or on the liquid medium, each of the plural receptacles arranged in fluid communication to allow for a recirculating flow of the liquid medium to cascade through the plural receptacles such that the recirculating flow of the liquid medium flows into at least one first receptacle of the plural receptacles via at least one inlet and flows out of at least one final receptacle of the plural receptacles via at least one outlet;
providing a sump containing an additional volume liquid medium, the sump having a sump inlet in fluid communication with the at least one outlet of the enclosed fluid network, and a sump outlet; and
operating a pump to govern a flow of the liquid medium between the sump outlet and the inlet of the enclosed fluid network to establish and/or maintain the recirculating flow of the liquid medium.

These and other embodiments of the present disclosure will become apparent to those of ordinary skill in the art, upon reviewing the following description of specific, exemplary embodiments of the present disclosure in conjunction with the accompanying figures. While features of embodiments of the present disclosure may be discussed relative to certain embodiments and figures below, all embodiments of the present disclosure may provide one or more of the advantageous features discussed herein. In other words, while one or more embodiments may be discussed as having certain advantageous features, one or more of such features may also be used in accordance with the various embodiments of the disclosure discussed herein.

### BRIEF DESCRIPTION OF DRAWINGS

Embodiments of the present disclosure will be discussed with reference to the accompanying drawings wherein:

FIG. 1 is a block diagram of a biomass reactor according to a first embodiment of the present disclosure;

FIG. 2 is a close-up cross-sectional view of a portion of a receptacle suitable for use with the biomass reactor shown in FIG. 1;

FIG. 3 is an isometric view of an example of a receptacle suitable for use with the biomass reactor shown in FIG. 1;

FIG. 4 is a schematic diagram of a receptacle arrangement suitable for use with a biomass reactor according to an embodiment of the present disclosure;

FIG. 5 is a schematic diagram of another receptacle arrangement suitable for use with a biomass reactor according to an embodiment of the present disclosure;

FIG. 6 is a perspective view of another receptacle arrangement suitable for use with a biomass reactor according to an embodiment of the present disclosure;

FIG. 7 is a cross-sectional view of the block diagram of the receptacle arrangement of FIG. 6;

FIG. 8 is a cross-sectional view of a sump suitable for use with a biomass reactor according to an embodiment of the present disclosure; and

FIG. 9 is a block diagram of a biomass reactor according to another embodiment of the present disclosure.

### DESCRIPTION OF EMBODIMENTS

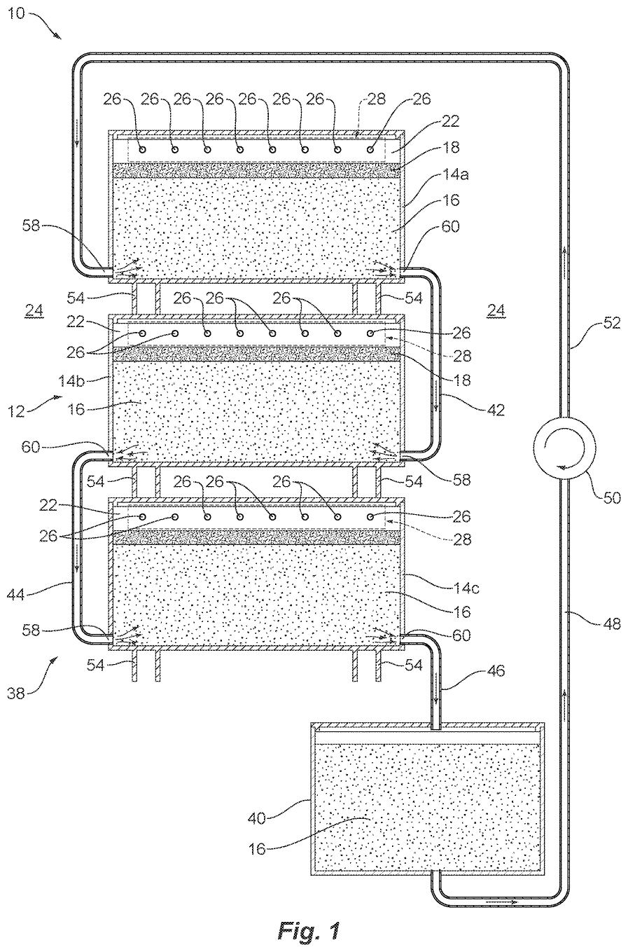

With reference to FIG. 1, there is illustrated a biomass reactor 10 (hereinafter ‘the reactor’) comprising an enclosed fluid network 12 including receptacles 14a, 14b, 14c. As shown, each receptacle 14a, 14b, 14c is configured to contain a volume of liquid medium 16 (hereinafter ‘the medium’) on or in which a microbial biomass 18 (hereinafter ‘the biomass’) is grown for production of molecular products such as compounds, nucleic acids, lipids and proteins.

Before continuing, throughout this specification references will be made to an “enclosed fluid network”. Where used in this specification, references to “enclosed fluid network” are to be understood to denote a network which supports direct or indirect fluid flow between elements of the network in a manner which, in normal operation, prevents unfiltered fluid flow (such as unfiltered air) from an external environment entering the elements of the network. In embodiments of the present disclosure, an enclosed fluid network allows contents of the receptacles (such as receptacles 14a, 14b, 14c) to be kept sterile from an external environment and thus reduces the risk of contaminating the medium 16 or the biomass 18 in the receptacles.

Throughout this specification references will also be made to the term “liquid medium”. Where used throughout this specification, references to the term liquid medium are to be understood to denote any aqueous solution or suspension of nutrients which is capable of sustaining the viability of the biomass 18 which floats on or is suspended or is immobilised in the liquid medium 16. Non-limiting examples of liquid media include Czapek-Dox, Potato Dextrose, Yeast extract+Sucrose (YeS), Lysogeny Broth (LB), Terrific Broth (TB), Soy-Peptone Sorbitol Broth (SPSB). It will be understood by a skilled person that certain liquid media are suitable for use in combination with one or more microbial biomasses.

In the context of this specification, references to the term “biomass” are to be understood to denote any biological cell-mass that produces, as a byproduct, molecular products that passively or actively leach into the liquid medium when the reactor is in use for extraction of the molecular product(s) therefrom. In this respect, non-limiting examples of suitable biomass include fungi (such as, Penicillium camemberti, Aspergillus nidulans, and Parastagonospora nodorum) and bacteria (such as, Escherichia coli, Vibrio natriegens, Bacillus subtilis, Streptomyces sp., and Anabaena azollae).

Throughout this specification, references will be also be made to a “receptacle”. Where used in this specification, references to “receptacle” are to be understood to denote any type of receptacle which is suitable for containing a volume of liquid medium capable of supporting growth of a biomass layer within the receptacle. Examples of suitable receptacles include trays, tanks, cups, containers, cans, bottles, flasks, bowls, pans, buckets or the like. The receptacles may or may not have a separate or removable lid or cover. In some embodiments of the present disclosure, the receptacles of the enclosed fluid network 12 are arranged or configured to provide an enclosed volume for containing the volume of the liquid medium 16 capable of supporting growth of the biomass layer 18 in each receptacle of the enclosed fluid network 12.

In the present case, the receptacles 14a, 14b, 14c are arranged in fluid communication to allow for a recirculating flow of the liquid medium 16 to cascade through the receptacles 14a, 14b, 14c of the enclosed fluid network 12 via sump 40, pump 50 and interconnecting tubes 42, 44, 46, 48, 52. In this respect, although the enclosed fluid network 12 of the reactor 10 shown in FIG. 1 comprises three receptacles 14a, 14b, 14c, it will of course be appreciated that a different number of receptacles may be arranged in fluid communication to form the enclosed fluid network 12. Furthermore, in certain embodiments the enclosed fluid network of the reactor 10 could comprise at least one receptacle in direct or indirect fluid communication with multiple downstream receptacles and/or multiple upstream receptacles.

In certain embodiments, the receptacles 14a, 14b, 14c include a stimulation element (not shown) for stimulating growth of the biomass 18. For example, each receptacle 14a, 14b, 14c could include a stimulation element that is formed integrally with, located in, or positioned proximal to each receptacle 14a, 14b, 14c to generate a stimulation output therein. It is possible that the stimulation element could be a suitably designed electronic module that is located in each receptacle 14a, 14b, 14c.

The characteristics of the stimulation element and thus the stimulation output may be selected, configured or tuned according to the biomass 18. Example stimulation outputs include vibration, acoustic, electric, magnetic, electromagnetic radiation (including infrared, ultraviolet, and gamma radiation), and ionising radiation. In one embodiment, the stimulation output is a photo-stimulation suitable for growing an algal biomass. In another embodiment, the stimulation output is a blue light having a wavelength suitable for triggering genetic cascades in a fungi biomass.

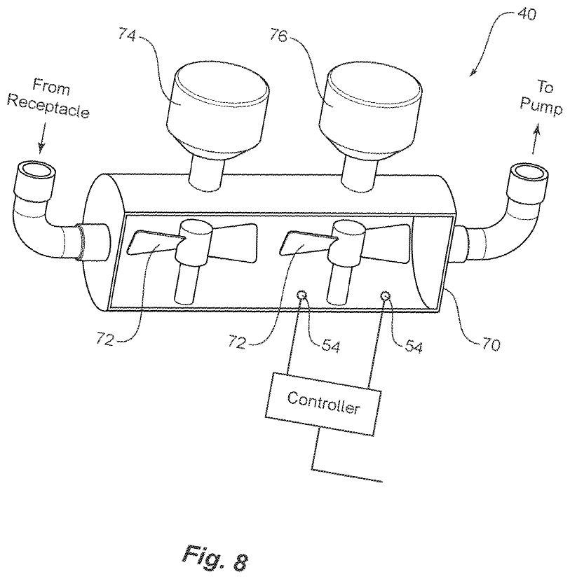

The sump 40 of the reactor 10 is a suitably sized vessel or container (such as a tank or jar) having a capacity to store a total volume of medium 16 which exceeds the total volume of medium 16 contained in the receptacles 14a, 14b, 14c, as will be further described below. In certain embodiments, the sump 40 is a separate vessel or container to the receptacles. However, it is possible that the sump 40 could be a lower most receptacle of the biomass reactor 10 having a suitable capacity. In one alternative embodiment, the sump 40 is a tube or pipe including static mixer structures. In this respect, FIG. 8 shows an example of a sump 40 in the form of a pipe 70 housing static mixer structures 72 (such as static vanes), sensing devices 78, fluid dispenser 74, and fluid feed 76. Fluid dispenser 74 may be a container or port for dispensing fluid into the pipe 70, such as may be required for pH balancing.

As shown in FIG. 1, sensing devices 78, such as temperature sensors, pH sensors, oxygen sensors, flow rate sensors or the like, may be used to sense properties of the liquid medium 16 (or other parameters of the process) in, flowing into or flowing out of the sump 40 for monitoring and/or control purposes.

As will be described in more detail below, when used to produce molecular products such as compounds, nucleic acids, lipids and proteins, a volume of the liquid medium 16 is provided and maintained within each receptacle 14a, 14b, 14c and the sump 40 by recirculating the liquid medium 16 at a particular flow rate through the enclosed fluid network 12 and thus the receptacles 14a, 14b, 14c. The particular flow rate is governed by the pump 50 which, in the present case, is a low-power peristaltic pump which will be described in more detail below. Tubes 42, 44, 46, 48, 52 may be rigid, semi-rigid or flexible tubes having a diameter which allows flow of the medium 16 therethrough at the particular flow rate.

The volume of liquid medium 16 in each receptacle 14a, 14b, 14c forms a layer for supporting a layer of biomass 18 within a respective receptacle 14a, 14b, 14c. Support of the layer of biomass 18 within a respective receptacle 14a, 14b, 14c by the layer of the liquid medium 16 may include the layer of biomass 18 floating on a surface of the liquid medium 16 or being at least partially suspended (i.e., partially immersed) in the liquid medium 16. For example, the biomass 18 may be supported on a suitable support structure (such as a supporting mesh or grid) in each receptacle 14a, 14b, 14c which prevents it from becoming completely submerged in the medium 16. In such embodiments, within each respective receptacle 14a, 14b, 14c an upper surface of the layer of biomass 18 is exposed to and interfaces with a volume of air 22 contained within each respective receptacle 14a, 14b, 14c of the enclosed fluid network 12 to permit passive diffusion of air to supply oxygen to the layer of the biomass 18 within a respective receptacle 14a, 14b, 14c. In other embodiments, the biomass 18 is completely immersed or submerged in the medium 16. For example, in some embodiments, the biomass 18 may sit on or towards the bottom of a receptacle 14a, 14b, 14c.

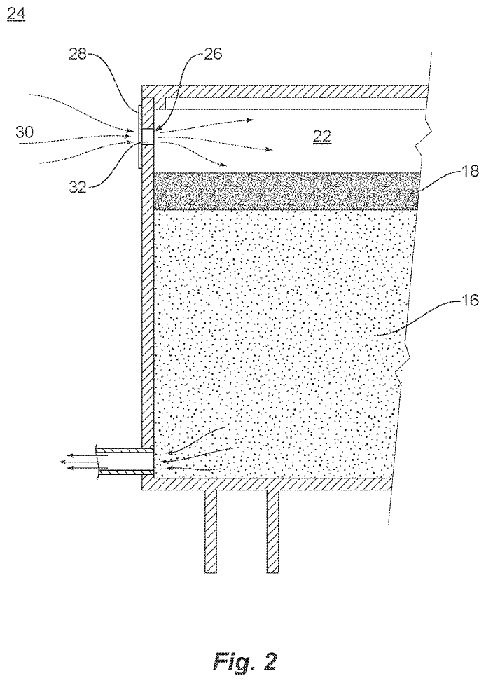

Continuing now with reference to FIG. 2, in the present case the volume of air 22 in each receptacle 14a, 14b, 14c is in fluid communication with an external air mass 24 via one or more air inlets 26.

In the present case, each air inlet 26 is obstructed or covered by a semipermeable membrane 28 (ref. FIG. 2) to form a semipermeable barrier between the volume of air 22 and the external air mass 24. The semipermeable membrane 28 allows air 30 from the external air mass 24 to permeate through each air inlet 26 and into the volume of air 22 contained within each receptacle 14a, 14b, 14c to passively diffuse across the layer of biomass 18. In this way, contaminants present in the external air mass 24 which may otherwise contaminate the interior volume of air 22, and thus the microbial biomass 18 and/or the liquid medium 16, are removed, at least to some extent, prior to the air entering receptacle 14a, 14b, 14c via an air inlet 26. An advantage of providing air inlets 26 which are arranged to allow for the above-described passive diffusion of air to supply oxygen to the layer of biomass 18 in a receptacle 14a, 14b, 14c is that mechanical failures and/or costs typically associated with stirring and aerating liquid medium 16 can be avoided. In particular, by providing a layer of biomass 18 having a suitable surface area and configuring the reactor 10 for passive diffusion of air, the need for mechanical aeration may be avoided. Furthermore, the combination of the surface area of the layer of biomass 18 and passive diffusion of air may also negate the need to use additional devices or means to dissipate heat. In this respect, large traditional reactors generate considerable heat which may be removed using cooling jackets which dramatically increases infrastructure and running costs.

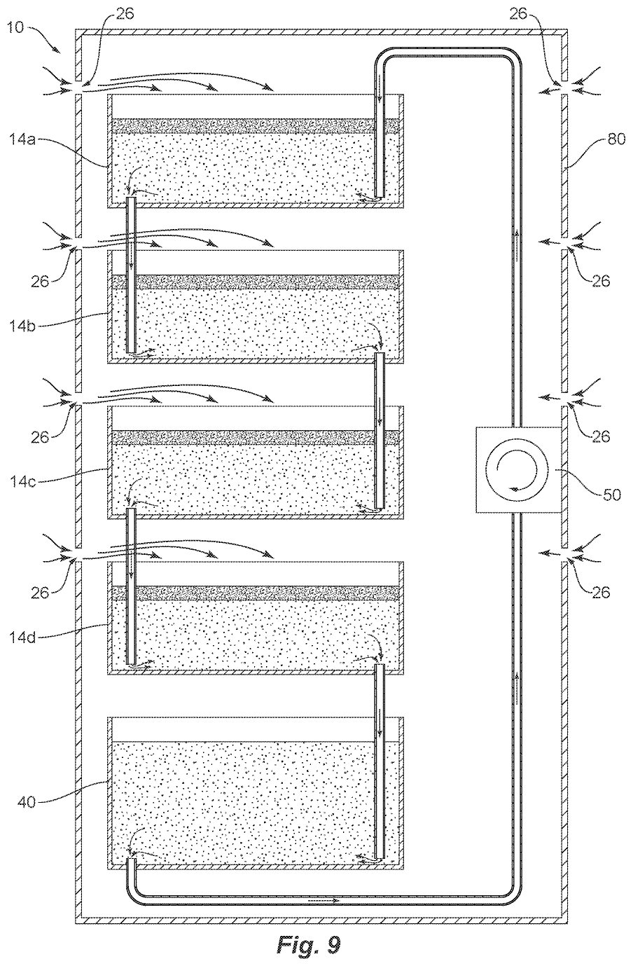

Although in the present case, each of the one or more air inlets 26 is an inlet of a respective receptacle, in a reactor (ref. FIG. 9) according to another embodiment, the receptacles 14a, 14b, 14c are located inside an enclosure 80, such as a shipping container, cabinet, box, compartment, or the like, and the one or more air inlets 26 are formed in the enclosure 80. In such an embodiment, air within the enclosure 80 may be inoculated, and air from outside of the enclosure 80 permeates through the semipermeable membrane (not shown) of each air inlet 26 to passively diffuse across the layer of biomass 18. In this way, contaminants which may otherwise contaminate the interior volume of air of the enclosure 80, and thus the microbial biomass 18 and/or the liquid medium 16, are removed, at least to some extent, prior to the air entering the enclosure 80.

Although the embodiment depicted in FIG. 9 includes a single vertical stack of receptacles, it is possible that the enclosure 80 may house plural vertical stacks of receptacles, with each stack having a separate respective pump 50 and suitable interconnections for allowing fluid communication between the receptacles of a stack. An advantage of an embodiment shown in FIG. 9 is that it provides a self-contained reactor 10 which is suitable for use at an industrial scale and which is readily scalable.

Returning now to FIG. 2, the semipermeable membrane 28 may be any suitable material which allows for permeation of air under normal atmospheric conditions, but which filters particles above a particular size. One example of a suitable semipermeable membrane material is surgical tape, such as Transpore™ surgical tape produced by 3M™. Other suitable semipermeable membrane materials would be well known to a skilled person. Non-limiting examples of other suitable semipermeable membrane materials include such 2 micron PETE (polyester track etched), poly carbonate track etched, polyethylene, and polyethersulfone (PES) nitrocellulose.

As described above, in the present case the semipermeable membrane 28 is positioned to obstruct a respective aperture 32 of each air inlet 26 to form the semipermeable barrier between the volume of air 22 in each receptacle 14a, 14b, 14c and the external air mass 24. Each aperture 32 thus defines the size of the air inlet 26 through which air may permeate from the external air mass 24, through the semipermeable membrane 28 into the internal volume of air 22 of a respective receptacle 14a, 14b, 14c to diffuse across and into the microbial biomass 18. Each air inlet 26 may have an associated single aperture 32 or it may comprise plural apertures 32.

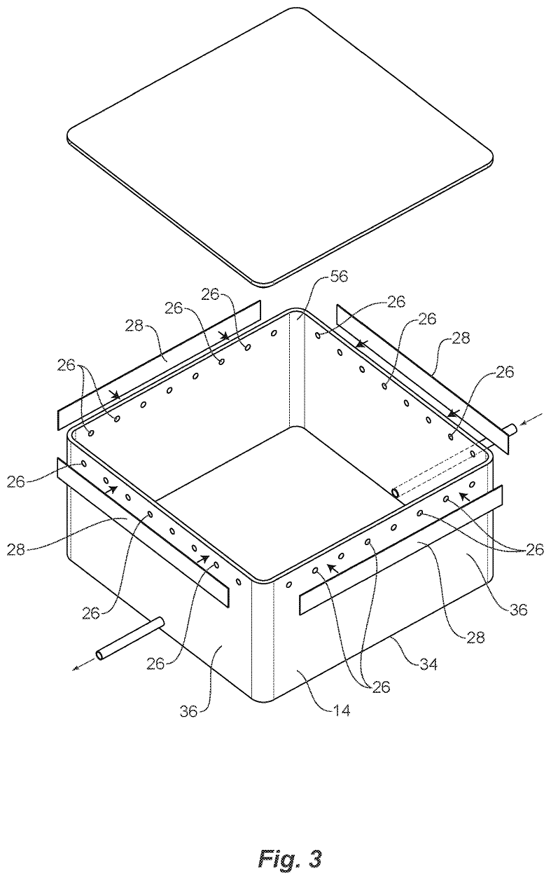

Turning now to FIG. 3 there is illustrated an embodiment of a receptacle 14 suitable for use as the receptacles 14a, 14b, 14c of the enclosed fluid network 12 depicted in FIG. 1. In the illustrated embodiment, receptacle 14 has a generally square base 34 and upstanding sidewalls 36. Each side wall 36 includes plural air inlets 26 covered by semipermeable membrane 28 in the form of lengths of semipermeable tape located over the plural inlets 26 of each side wall 36. A removable lid 56 sealably engages with a top rim of the receptacle 14. The removable lid 56 may allow access to the interior of the receptacle 14 for cleaning and/or sterilising the receptacle 14. In the present case, each receptacle 14a, 14b, 14c of the enclosed fluid network 12 depicted in FIG. 1 has the same size and shape as the receptacle shown in FIG. 2.

Before continuing further, although in the present case, the size and shape of each receptacle 14a, 14b, 14c of the enclosed fluid network 12 is the same, it is not essential that receptacles 14a, 14b, 14c have the same shape and size. Indeed, it is anticipated that other embodiments of the reactor 10 may involve the use of receptacles having a different size and shape. For example, in some embodiments the size and shape of each receptacle of a reactor 10 may be individually selected to provide a volume (V), depth (D) or side wall perimeter configuration optimised for growth of a particular microbial biomass. In other embodiments, the size and shape of each receptacle may be selected to provide volumetric flow characteristics, such as a flow distribution within the volume of the liquid medium 16 in the receptacle, which provides for improved microbial biomass growth, by providing, for example, a particular pressure head.

As will be appreciated, there is a relationship between flow rate, metabolism, and volume. For example, as the surface area of a receptacle 14 increases, so does the metabolism and nutrient requirements of the layer of biomass 18, requiring a faster flow rate to ensure the biomass 18 is maintained. Suitable flow rates thus depend on the size of the reactor 10 in terms of receptacle diameter as well as total system volume. A suitable flow rate for a 1 to 10 litre reactor comprising 30 cm×30 cm trays may be between 50 ml and 300 ml per minute.

It is also important, but not critical, to maintain the total volume in all receptacles 14a, 14b, 14c at less than the volume of the sump 40. In this respect, if the total volume in all receptacles 14a, 14b, 14c exceeds the volume of the sump 40, and because the receptacles 14a, 14b, 14c drain into the sump 40 (as the sump 40 is simultaneously pumped back into the top receptacle 14a), if the pump 50 is switched off or fails, the sump 40 could overflow or cause backpressure.

Returning again now to FIG. 1, and as outlined above, in the present case enclosed fluid network 12 comprises three receptacles 14a, 14b, 14c arranged and connected in fluid communication to allow a recirculating flow of the liquid medium 16 to cascade through each receptacle 14a, 14b, 14c of the network 12. In the present case, a cascading flow is achieved by arranging the receptacles 14a, 14b, 14c as a vertically offset stack to form a tower 38 in which the liquid medium 16 flows generally downwardly from receptacle 14a, and through receptacle 14b and 14c in the direction of sump 40.

In the illustrated embodiment, the tower 38 is formed by mounting receptacle 14a on top of receptacle 14b which in turn is mounted on top of receptacle 14c. In the present case, supports 54 are used to mount the receptacles to form the tower 38. However, it is possible that receptacles 14a and 14b may be mounted or stacked directly to receptacles 14b and 14c respectively. An example of suitable supports 54 includes “stand offs” or spacers which are configured and positioned to provide a gap therebetween.

The supports 54 may be formed integrally with the base 34 (ref. FIG. 3) of a receptacle 14, for example, or they may be formed as or part of a separate post, frame or rib which is interposed between the receptacles 14a, 14b, 14c. Providing a gap between the receptacles 14a, 14b, 14c may allow air inlets 26 to be distributed across an upper surface of each receptacle 14 as opposed to just the side walls 34 (ref. FIG. 3). An advantage of distributing the air inlets 26 across an upper surface of each receptacle 14 is that it may allow for a more uniform distribution airflow by reducing the average distance between points on the surface of the liquid medium 16 and the nearest air inlets 26.

In operation, the volume of the liquid medium 16 in the receptacles 14a, 14b, 14c can be maintained in a steady state by pumping medium 16 into the upper most (or first) receptacle 14a at the same flow rate as lower most receptacle 14c drains into the sump 40, and by maintaining a constant input during initiation of the reactor 10. In this mode, liquid medium 16 contained in sump 40 is continuously pumped from the sump 40 via tube 48 using pump 50 and into receptacle 14a via tube 52 to establish a continuous recirculating flow of the liquid medium 16 which cascades through each receptacle 14a, 14b, 14c of the enclosed fluid network 12. However, it is also possible that the reactor 10 could be run in a “pulsed” mode or cascade effect whereby one receptacle drains into the next and triggers it to flow. In this mode, a draining receptacle 14 will stop dispensing medium 16 until it is next “triggered”. This continues down the stack, with one receptacle 14 triggering the next and then stopping until the medium 16 is recycled to the top of the receptacle stack.

Continuing with reference to FIG. 1, each receptacle 14a, 14b, 14c includes at least one fluid inlet 58 and at least one fluid outlet 60. In this respect, although in the present case each receptacle 14a, 14b, 14c is depicted with a single fluid inlet 58 and a single fluid outlet 60 in other embodiments, each receptacle 14a, 14b, 14c may include plural fluid inlets 58 and/or a plural fluid outlets 60.

As shown in FIG. 1, each fluid inlet 58 and fluid outlet 60 is located below the layer of biomass 18 in the medium 16. This arrangement allows the medium 16 to flow freely without disturbing the layer of biomass 18 floating on or suspended in the medium 16. In this respect, during commissioning of the reactor 10, as each receptacle 14a, 14b, 14c is filled with a volume of the medium 16, beginning with the upper most receptacle 14a, when the medium 16 reaches a certain height in each receptacle 14, the receptacle's 14 outlet 60 will activate and dispense the medium 16 into the next receptacle 14 or receptacles 14 in the fluid network 12, which in turn will fill and dispense. The final layer (which in the illustrated example is the layer of medium 16 in receptacle 14c) dispenses into a device (not shown) for extraction of high value components secreted by the layer of biomass 18. For example, the medium 16 from receptacle 14c may flow through a resin that extracts non-polar medium molecular weight compounds, or a protein affinity column to collect proteins of interest from the medium 16. The medium 16 is then passed to a sterile container, shown here as sump 40, to be replenished. In this respect, replenishment includes pH, osmolyte, and oxygen correction as well as nutrients. Once replenished, the medium 16 is passed to the top receptacle 14a to repeat the cycle.

In the present case, each fluid inlet 58 and fluid outlet 60 of a receptacle 14a, 14b, 14c is disposed in opposite sidewalls 36 and generally towards the base 34 of each receptacle 14a, 14b, 14c to provide a vertical separation(S) between the height of the fluid inlet 58 and fluid outlet 60 and the height of the layer of biomass 18. In preference, fluid inlet 58 and fluid outlet 60 are positioned proximal to the base 34 to maximise the vertical separation(S).

Providing a vertical separation between the height of the fluid inlet 58 and fluid outlet 60 and the height of the layer of biomass 18 may reduce disturbance of the layer of biomass 18 which could otherwise result from flow of liquid medium 16 into a receptacle 14a, 14b, 14c via fluid inlet 58 and/or the flow out of a receptacle 14 via fluid outlet 60. Such disturbance may otherwise have a detrimental impact on growth of the biomass 18. In effect, in embodiments, the layer of biomass 18 can be grown in near steady state conditions, which allows the layer of biomass 18 to use incoming nutrients to produce the desired molecules without requiring the biomass 18 to be killed. Furthermore, disposing each fluid inlet 58 and fluid outlet 60 generally towards the base 34 of each receptacle 14a, 14b, 14c potentially allows for a relatively shallow depth of the liquid medium 16 within each receptacle 14a, 14b, 14c. In this respect, a shallow medium 16 layer reduces wasted space in each receptacle 14 and thus may allow for thicker growth of the biomass 18 layer.

During growth of the layer of biomass 18, the viscosity of the medium 16 may increase as the composition of the medium 16 changes, including as the biomass 18 secretes molecules into the medium 16. As the viscosity of the medium 16 increases, the meniscus of the medium 16 may also change and alter how the medium 16 interacts with the fluid inlets 58, fluid outlets 60 and tubes 42, 44, 46, 48 and 52. For example, during operation, changes in the meniscus of the medium 16 may impact on the flow of the medium 16 from the fluid outlets 60.

To maintain consistent flow, the diameters of the fluid inlets 58, fluid outlets 60 and tubes 42, 44, 46, 48 and 52 should be selected to have a minimum diameter which supports fluid flow across the expected range of viscosity of the medium 16. In this respect, the internal diameter of the fluid inlets 58, outlets 60, and tubes 42, 44, 46, 48 and 52 should be wide enough to avoid the meniscus trapping bubbles in the tubes 42, 44, 46, 48 and 52, and reduce the risk of flow rates generating a jet of the medium 16 that could disturb the biomass 18. In embodiments, tubing having an internal diameter of between 15 mm and 20 mm may be used. However, a larger diameter may be required as flow rate increases to match the increased biomass 18. It will be appreciated that the minimum diameter will thus depend on the properties of the medium 16 and, in particular, the viscosity of the medium 16 as the composition of the medium 16 changes. It will also be appreciated that the minimum diameter of the fluid inlets 58 and outlets 60 places constraints on the minimum depth of the receptacle 14.

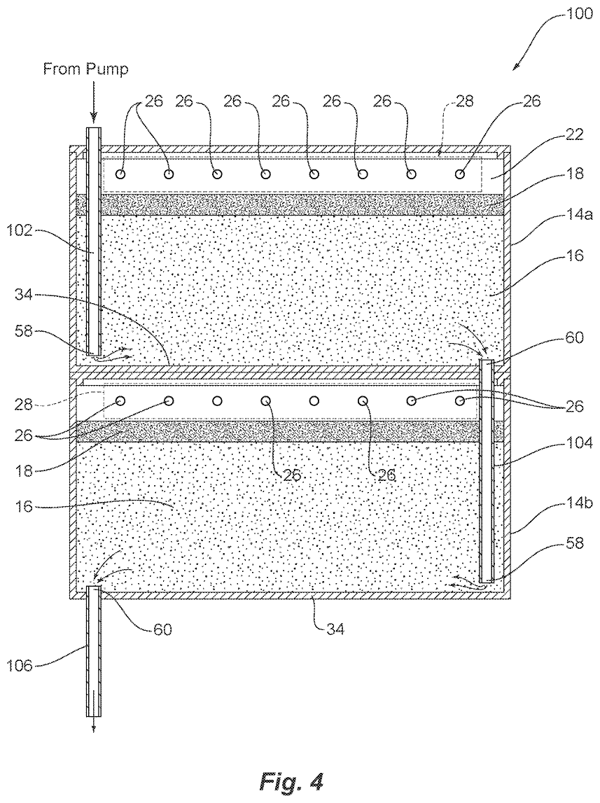

Turning now to FIG. 4 there is shown another example of a receptacle arrangement 100 suitable for use with embodiments of the present disclosure. For clarity, only two receptacles 14a, 14b are depicted although it will of course be appreciated that additional receptacles may be used. In the receptacle arrangement 100 of FIG. 4, receptacle 14a is located on and supported by receptacle 14b. Receptacles 14a, 14b include the same configuration of air inlets 26 as described above in relation to FIGS. 1 to 3. Tubes 102, 104, 106 are equivalent in function to tubes 52, 42, 44 of FIG. 1. However, in the receptacle arrangement 100 of FIG. 4, tubes 102, 104, 106 are arranged vertically to provide, in this example, downwardly depending fluid inlets 58 and fluid outlets 60. It will of course be appreciated that other inlet 58 and outlet 60 arrangements may be used. For example, it is possible that the inlet 58 and outlet 60 arrangements may be configured or shaped to create a flow pattern which projects off a vertical axis.

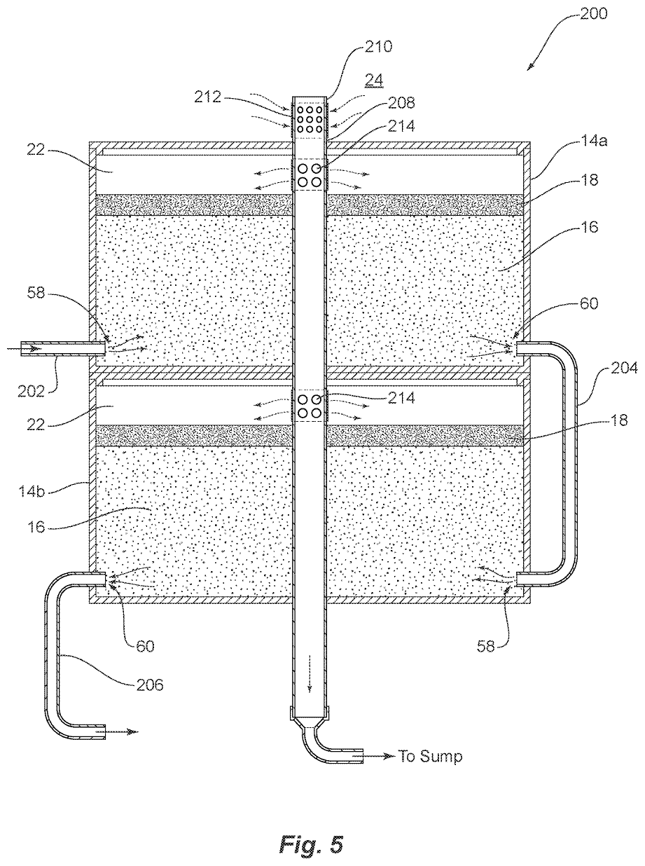

With reference now to FIG. 5 there is shown another example of a receptacle arrangement 200 suitable for use with embodiments of the present disclosure. For clarity, only two receptacles 14a, 14b are depicted although it will of course be appreciated that additional receptacles may be used.

In the receptacle arrangement 200 of FIG. 5, receptacle 14a is located on and supported by receptacle 14b. Tubes 202, 204, 206 are equivalent in function to tubes 52, 42, 44 of FIG. 1. The receptacle arrangement 200 has a central channel in the form of a tube 208 which extends vertically and centrally through receptacles 14a, 14b. Tube 208 includes a portion 210 (shown here as a top portion) having one or more air inlets 212 which are obstructed or covered by semipermeable membrane 28. Air may permeate from the external air mass 24 through the semipermeable membrane 28 covered air inlets 212 and into the internal volume of air 22 of each receptacle 14a, 14b via one or more air inlets 214. Tube 208 is thus configured such that contaminants which may otherwise contaminate the interior volume of air 22, and thus the biomass 18 and/or the liquid medium 16, are removed, at least to some extent, prior to the air entering receptacles 14a, 14b. In the receptacle arrangement 200 of FIG. 4, tube 208 has a connection to sump 40 (ref. FIG. 1). An advantage of the receptacle arrangement of FIG. 4 is that the air inlets 214 also allow for flow of fluid from a receptacle 14a, 14b into the sump 40 if the total volume of medium 16 and/or biomass 18 in a receptacle 14a, 14b exceeds a threshold level. In other words, the air inlets 214 may be positioned to allow air to permeate from the external air mass 24 into the internal volume of air 22 and also reduce the risk of a receptacle 14 overflowing. Although not shown in FIG. 5, it is possible that receptacles 14a, 14b could additionally include air inlets 26 of the type described in relation to FIGS. 1 to 4.

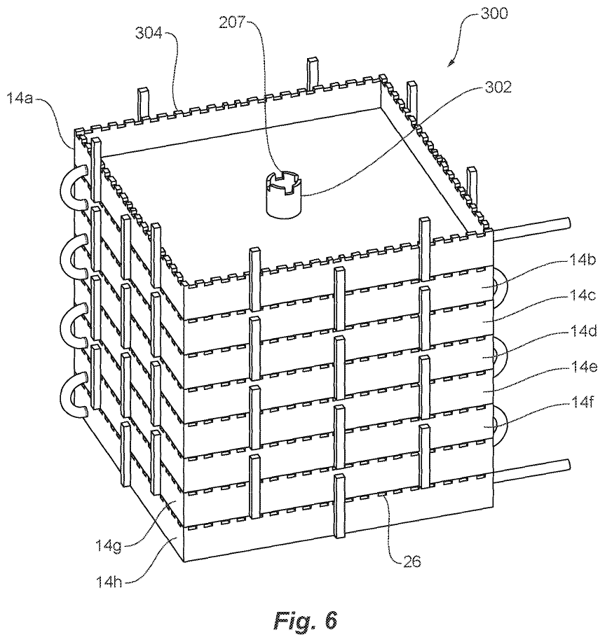

Continuing now with reference to FIG. 6, there is shown another example of a receptacle arrangement 300 suitable for use with embodiments of the present disclosure. The receptacle arrangement 300 has eight receptacles 14a, 14b, 14c, 14d, 14e, 14f, 14g, 14h. In this embodiment, each receptacle 14 includes plural air inlets 26 of the type described above in relation to FIGS. 1 to 4. When assembled as a stack of vertically offset receptacles 14, a central channel 207 similar to the central tube 208 described in relation to FIG. 5 is formed by connecting tubular connectors 302 formed integrally with each receptacle 14a, 14b, 14c, 14d, 14e, 14f, 14g, 14h.

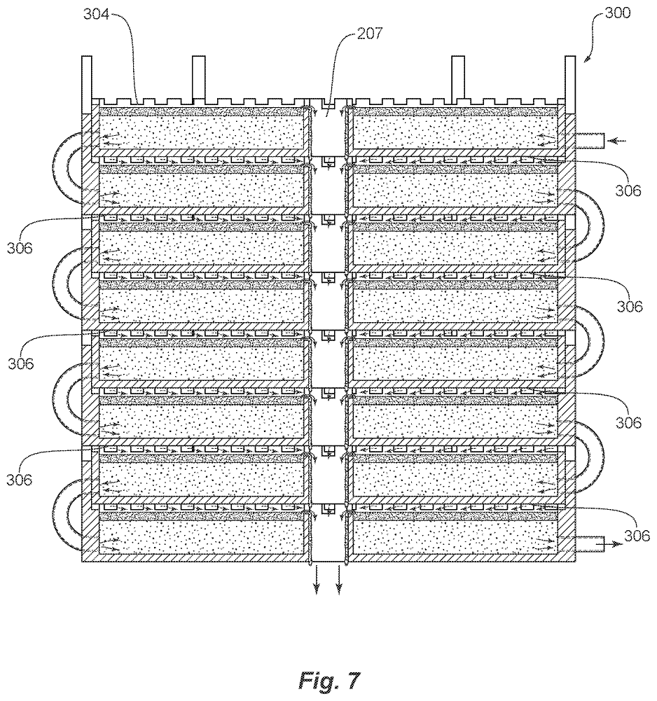

As shown in FIG. 7, in receptacle arrangement 300, receptacles 14a, 14b, 14c, 14d, 14e, 14f, 14g, 14h have a serrated upper rim 304 which is contactable by an underside surface 306 seated on top of the serrated upper rim 304 to form the plural air inlets 26 (ref. FIG. 6). Surface 306 could be the base 34 (ref. FIG. 3) of a receptacle 14 seated on top of the serrated upper rim 304 of a lower receptacle, or a lid, depending on the position of the receptacle 14 in the arrangement 300.

An advantage of embodiments of the present disclosure is that the disclosed reactor is able to run without aeration, and not be in danger of contamination, due to the large surface area of the biomass in contact with the air. As described above, in embodiments the receptacles are separated from the open air by a semipermeable membrane which allows gasses to diffuse into a receptacle in a way which blocks contaminants. In this way, oxygen can diffuse across the medium 16 and over the biomass 18. As a result, there is no need to oxygenate the medium 16 thus reducing cost, risk of contamination, and risk of degrading the high-value products.

It will be appreciated from the foregoing that a range of molecular products that are ordinarily produced by a biomass can be produced using the reactor and separated from the medium 16 for further use. Molecular products that are ordinarily produced by bacterial, fungal or algal biomass include small molecule compounds, nucleic acids, amino acids, peptides and proteins. The molecular products may be primary metabolites of the bacterial, fungal or algal species present in the biomass or they may be secondary metabolites (i.e. derivatives of primary metabolites). Advantageously, the molecular products produced may be of high value or of commercial value, such as drugs, active ingredients, or fermented products.

If desired, the molecular products may be separated from the liquid medium or isolated from the liquid medium using known isolation techniques such as precipitation, liquid-liquid extraction (SX), liquid-solid extraction, chromatography, etc.

Other embodiments of, and application for, the reactor 10 are also contemplated. One embodiment involves liquid-phase solvent extraction in which the medium 16 is pumped over a well, such as a chloroform well, prior to flowing into the sump 40. In this example, the molecule of interest is a molecule which is more soluble in chloroform than water and so accumulates in the chloroform. The molecule of interest is then extracted and purified from the chloroform.

Another embodiment may be used for reverse-phase chromatographic extraction. In such an embodiment which the medium is 16 pumped through a resin that captures the molecule of interest. The resin is packed into a tube disposed between the final outlet of the tank arrangement and the sump as fine powder like beads. The tube may be connected using an arrangement which allows the tube to be quickly removed and a fresh tube inserted while the reactor is running. Products of the reactor can then be washed from the beads with solvent, refreshing the beads, and the tube returned to the system.

Further references to operational aspects of the reactor 10 will now be described in the below non-limiting examples.

### Example 1: Commissioning

With reference to FIG. 1, a reactor 10 according to an embodiment of the present disclosure may be commissioned for operation by a suitable commissioning process. In one example of a suitable commissioning process, and with reference to FIG. 1, receptacles 14a, 14b 14c are sterilised with alcohol in a sterile chamber. Interconnecting tubing 42, 44, 46 are sterilised with alcohol and connected as shown in FIG. 1. The receptacles 14a, 14b, 14c are filled with an inoculated medium 16 to a minimal volume required to cover the inlets 58 and outlets 60. The receptacles 14a, 14b, 14c are assembled on top of each other to form the depicted stacked arrangement in which the receptacles 14a, 14b, 14c are vertically offset. Tubing 46, 48 to the filter (not shown), sump 40, and pump 50 is washed with alcohol and then connected to the stack of receptacles 14a, 14b, 14c.

The sump 40 is partially (for example, half) filled with sterile medium 16. Reactor 10 is then removed from the sterile chamber and left to incubate at room temperature for several days until the biomass 18 has covered the surface of the medium 16.

Pump 50 is then activated and sterile medium 16 from sump 40 is pumped into the top receptacle 14a of the reactor 10. Once the volume of medium 16 in the top receptacle 14a has reached a certain volume, the outlet 60 of receptacle 14a is triggered and the second receptacle 14b begins to fill via its inlet 58. This process is repeated for receptacles 14b and 14c.

The reactor 10 is run and biomass is established. The reactor 10 continues to run until the biomass begins to deteriorate. At that point, the filter (not shown) is replaced and molecular product(s) are collected as needed.

The pH and dissolved oxygen are monitored and corrected as needed (high oxygen indicates low metabolic activity so more nutrients are needed or the system is dying). The medium 16 in its entirety could be replaced while the reactor 10 is running. For example, if the reactor 10 has been running for an extended period and there is a build of salts in the medium 16, the pump 50 could be connected to a new sump 40 containing fresh medium 16, and the previous sump 40 filled with the old medium 16 discarded.

### Example 2: Syncytium Production

A biomass 18 was established with Potato Dextrose Broth [Sigma] in 1 litre receptacles 14, and a 100 ml of a 20-times concentrate of Czapek Dox [Sigma] medium was injected into the sump 40 once the reactor 10 was running using a 100 ml/minute Grothen G328 12 v peristaltic pump 50.

Medium 16 flowed into a chloroform wash and into the sump 40. In the sump 40, the medium 16 was stirred, and the pH, ionic strength, and dissolved oxygen were measured. Every 12 hours the pH was corrected with a solution of sodium phosphate salts and sodium hydroxide.

The biomass 18 was established after 48 hours, forming a 5 mm thick mass of white mycelium that covered the entire surface of the medium 16 in each receptacle 14a, 14b, 14c. The biomass 18 was not friable and maintained integrity throughout the run at 22° C. Without biomass 18, the bioreactor 10 aerated itself to 8.7 mg/litre—the maximum for that temperature. With biomass 18, the medium 16 was depleted of oxygen (1.5 mg/l) and the air directly provided oxygen to the biomass 18.

The reactor 10 produced 71 mg of product which was 10.5 fold more efficient than comparable methods measure in volume per time (i.e., litres per day of the reactor).

### Example 3: Compound Production

Sterile vermiculite [Brunnings™] and SFM medium (20 g/l Sorbitol [Sigma™ cat. 85529], 20 g/l Soy flour [Lotus™]) filled four 30 ml receptacles 14 of a reactor 10 according to an embodiment. Each receptacle held 10 ml of liquid medium 16, and the remaining space was filled loosely with vermiculite.

The medium 16 was inoculated with Streptomyces sp. spores and incubated at 25° C. After seven days, a biofilm had formed a cohesive mat, incorporating the vermiculite. The reactor 10 was then run in a pulsed mode with a four-day period whereby the liquid medium 16 was drained from the reactor 10 and passed through a fresh cellulose column (10 g Avicel™ PH-101, [Sigma™ cat. 11365]), before 40 ml of fresh SFM medium was supplied to the reactor 10. The reactor 10 was run for six cycles, producing ca. 120 ml of spent liquid medium. The biomass 18 in the reactor 10 was still viable and growing at the time the experiment ended.

The cellulose columns were washed with two volumes of MilliQ water, then two volumes of methanol at a rate of 1 ml per minute. The methanolic fractions were then combined and stored at −20° C. overnight precipitating a white fluffy complex which was then filtered. The filtered methanolic fraction was spiked with an internal standard of 0.2 mg/ml caffeic acid [Sigma™ cat. C0625] by aliquoted 50 μl of the fraction into a tube containing 50 μl of the caffeic acid solution. The resulting 100 μl aliquot was then dried under vacuum at room temperature. This fraction was then eluted in 50:50 acetonitrile and water for comparison with a purified Niphimycin cocktail, via High Performance Liquid Chromatography. The methanolic extract, cleaned of white precipitate, contained a Niphimycin cocktail of similar purity as to the purified standard generated via column chromatography.

### Example 4: Fungal Production of Multiple High and Low Value Molecules

Under sterile conditions, six one-litre receptacles 14 were assembled to form a reactor 10 having a configuration similar to the reactor 10 depicted in FIG. 1, but with six receptacles 14. The receptacles 14 were stacked and connected with valved tubing to control flow between the receptacles 14.

The valves were closed, and each receptacle 14 of the stack was filled with one litre of liquid medium containing 100 g of sucrose [Sigma™ cat. S0389], 100 g of yeast extract [Sigma™ cat. 09182] and P. citrinum strain: 5352 spores. The receptacles 14 were incubated at room temperature for three days while a mycelial mat biomass formed across the surface of the medium. Once the mycelial mat had formed, the final or lower most receptacle 14 in the stack was connected to a sump 40 that could hold the total volume of the reactor 10.

The sump 40 was connected to a tube containing a static mixer, pH probe and dissolved oxygen probe, which was connected to nine resin columns in parallel. The columns contained 50 g of Diaion™ HP-20 resin [Sigma™ cat. 13607] and were then connected to a peristaltic pump 50 which fed into the fluid inlet of the first or upper most receptacle 14 of the stack. The pump 50 was run at 100 ml per minute for 5 days, and pH and dissolved oxygen were monitored. The pump 50 and monitoring equipment ran from three 9 Volt batteries per day, using 0.37125 kilojoules per litre per hour. At the end of five days, the resin columns were removed and washed with one volume of distilled water, then two volumes of methanol and one volume of chloroform. The organic fractions were combined, and 50 μl was spiked with caffeic acid [Sigma™ cat. C0625] to a final concentration of 0.2 mg/ml and analysed by LCMSMS. This analysis identified citrinin and dihydrolysergic acid, at culture concentrations of 830 mg/l, and 9 mg/l, respectively.

### Example 5: Bacterial Protein Production

Streptomyces sp. was grown on minimal medium in a reactor 10 according to an embodiment of the present disclosure operating in a pulsed mode as described above.

The minimal medium contained sorbitol g/l Sorbitol [Sigma™ cat. 85529] 10 g/l and dextrin [Sigma™ cat. 31400] 10 g/l, sodium nitrate 1 g/l [Sigma™ cat. S5506], trace elements, and was buffered to pH 7.0 with 10 mM phosphate. After three days 37.5 ml of medium was collected from the reactor 10 and a 50 ul aliquot was passed through Sephadex g-10 [Sigma™ cat. G10120] to remove low molecular weight impurities.

Nanodrop spectroscopy then determined the protein content to be 960 mg/l indicating that the reactor 10 had produced 320 mg/l/day of total secreted protein

### Example 6: Fermented Beverage Production—Continuous Beer Production

Under sterile conditions, four 200 ml flasks were filled with 50 ml of liquid medium containing 20 g/l sucrose [Sigma™ cat. S0389], 5 g/l yeast extract [Sigma™ cat. 09182] and a commercial ale yeast.

The cultures were incubated at 30° C. and 150 RPM for 48 hours. These cultures were then combined and evenly distributed among four one-litre receptacles 14. The receptacles 14 were then stacked and connected via silicone tubing to assemble a reactor 10 configuration of the general type described above with reference to FIG. 1, but with four receptacles 14 in the form of trays.

Each receptacle 14 was filled with one litre of liquid medium containing 1 gram of dried brewers' hops and 100 g of dried dark malt extract. The starting specific gravity of the medium was 1.045. The receptacles 14 were then sealed with plastic tape and incubated at room temperature for three days. The first or upper most receptacle 14 was then connected to a peristaltic pump feeding in more malt-hops liquid medium from a one-litre bottle at 0.5 ml/minute, and the bottom outlet was connected to an empty one-litre bottle. Liquid medium draining from the reactor 10 contained no yeast sediment, smelled of hops, malt, and alcohol, and had a specific gravity of 1.025, indicating an alcohol content of 2.63%.

### Example 7: Fermented Beverage Production—Continuous Production of Kombucha

A Kombucha starter culture was homogenised at high speed in a kitchen blender and evenly distributed among four one-litre receptacles 14 in the form of trays.

The receptacles 14 were then stacked and connected via silicone to assemble a reactor 10 configuration of the general type described above with reference to FIG. 1, but with four receptacles 14. Each receptacle 14 was filled with one litre of liquid medium containing 2 g of dried green tea [Lipton™], 80 g of sucrose [Sigma™ cat. S0389], and 5 g of yeast extract [Sigma™ cat. 09182]. The receptacles 14 were then incubated at room temperature for 14 days, before being drained and refilled with tea-sucrose medium as described, but without yeast extract. The starting pH was 7.0. The first or upper most receptacle 14 was then connected to a peristaltic pump feeding in tea-sucrose liquid medium from a one-litre bottle at 0.5 ml/minute, and the bottom outlet was connected to an empty one-litre bottle. The liquid medium draining from the reactor contained no bacterial pellet, smelled of acetic acid, and had a pH of 4.2 indicating that the tea-sucrose medium had fermented into the acidic Kombucha beverage.

### Example 8: Waste Product Removal or Valorisation

Liquid medium containing 10 g/l ethylene glycol [Sigma™ cat. 102466], 1 g/l sodium nitrate [Sigma™ cat. S5506], and trace elements, was buffered to pH 6.5 with 10 mM phosphate buffer and sterilised. Once sterilised, 500 ml of this medium was inoculated with Aspergillus niger spores and dispensed evenly between five 100 ml receptacles 14 in the form of trays.

The receptacles 14 were then connected to assemble a reactor 10 configuration of the general type described above with reference to FIG. 1, but with five receptacles 14, and incubated at room temperature. The receptacles 14 were pulsed such that the medium was refreshed every three days. After 14 days, a thick mycelial mat had formed across the surface of the trays, measuring 8 mm in depth, indicating that this fungus was able to metabolise and assimilate this toxic waste product into fungal biomass.

The reference to any prior art in this specification is not, and should not be taken as, an acknowledgement or any form of suggestion that such prior art forms part of the common general knowledge.

It will be understood that the terms “comprise” and “include” and any of their derivatives (e.g. comprises, comprising, includes, including) as used in this specification, and the claims that follow, is to be taken to be inclusive of features to which the term refers, and is not meant to exclude the presence of any additional features unless otherwise stated or implied.

In some cases, a single embodiment may, for succinctness and/or to assist in understanding the scope of the disclosure, combine multiple features. It is to be understood that in such a case, these multiple features may be provided separately (in separate embodiments), or in any other suitable combination. Alternatively, where separate features are described in separate embodiments, these separate features may be combined into a single embodiment unless otherwise stated or implied. This also applies to the claims which can be recombined in any combination. That is a claim may be amended to include a feature defined in any other claim. Further a phrase referring to “at least one of” a list of items refers to any combination of those items, including single members. As an example, “at least one of: a, b, or c” is intended to cover: a, b, c, a-b, a-c, b-c, and a-b-c.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A biomass reactor, comprising:
an enclosed fluid network including plural receptacles, each receptacle for containing a volume of a liquid medium for growing a microbial biomass in or on the liquid medium,
wherein the plural receptacles are arranged in fluid communication to allow for a recirculating flow of the liquid medium to cascade through the plural receptacles.

2. A biomass reactor according to claim 1 wherein each receptacle includes plural air inlets, each air inlet being associated with at least one aperture which is covered or obstructed by a semipermeable membrane permitting passive diffusion of air into the internal volume from an external environment via the plural air inlets.

3. A biomass reactor according to claim 2 wherein the air inlets are uniformly disposed across:
an upper surface of each receptacle; or
one or more sidewalls of each receptacle; or
an upper surface and one or more sidewalls of each receptacle.

4. A biomass reactor according to claim 1 wherein the recirculating flow of the liquid medium flows into at least one first receptacle of the plural receptacles via at least one inlet and flows out of at least one final receptacle of the plural receptacles via at least one outlet, and
wherein the at least one first receptacle and the at least one final receptacle are in direct fluid communication or in indirect fluid communication involving one or more other receptacles.

5. A biomass reactor according to claim 4 wherein each receptacle of the plural receptacles has at least one respective inlet and at least one respective outlet, and
wherein the respective at least one inlet of the at least one first receptacle receives a flow of the liquid medium from a pump of the enclosed fluid network and the respective at least one outlet of the at least one final receptacle drains liquid medium into a sump of the enclosed fluid network, and
wherein the sump is in fluid communication with the pump so that the pump can pump liquid medium from the sump into the at least one first receptacle.

6. A biomass reactor according to claim 4 wherein the at least one inlet and the at least one outlet of each respective receptacle is juxtaposed to a base of each respective receptable.

7. A biomass reactor according to claim 1 wherein the plural receptacles are arranged as a multistage network, wherein each stage comprises at least one receptacle.

8. A biomass reactor according to claim 1 further comprising a layer of biomass disposed on a surface of the liquid medium contained in each receptacle of the enclosed fluid network.

9. A biomass reactor according to claim 4, further comprising a layer of biomass disposed on a surface of the liquid medium contained in each receptacle of the enclosed fluid network wherein for each receptacle, the respective inlet and outlet are configured to reduce disturbance of the biomass layer during the recirculating flow of the liquid medium.

10. A biomass reactor according to claim 1 wherein the plural receptacles are arranged as a vertically offset stack of receptacles.

11. A biomass reactor, comprising:
an enclosed fluid network including plural receptacles, each receptacle containing a volume of a liquid medium and a microbial biomass growing in or on the liquid medium, each of the plural receptacles arranged in fluid communication to allow for a recirculating flow of the liquid medium to cascade through the plural receptacles such that the recirculating flow of the liquid medium flows into at least one first receptacle of the plural receptacles via at least one inlet and flows out of at least one final receptacle of the plural receptacles via at least one outlet;
a sump containing an additional volume liquid medium, the sump having a sump inlet in fluid communication with the at least one outlet of the enclosed fluid network, and a sump outlet; and
a pump for generating a flow of the liquid medium between the sump outlet and the inlet of the enclosed fluid network to establish and/or maintain the recirculating flow of the liquid medium.

12. A biomass reactor according to claim 11 further comprising:
one or more sensors for sensing one or more parameters of the liquid medium flowing from the at least one final receptacle; and
means for making a correction to the one or more sensed parameters depending on the sensing.

13. A method for producing a molecular product, comprising:
providing an enclosed fluid network comprising plural vertically offset receptacles arranged in fluid communication, each receptacle containing a volume of a liquid medium for growing a microbial biomass in or on the liquid medium;
establishing fluid communication of the liquid medium between at least one final receptacle of the enclosed fluid network and at least one first receptacle of the enclosed fluid network so as to provide a recirculating flow of the liquid medium through the plural receptacles of the enclosed fluid network; and
processing liquid medium obtained from the at least one final receptacle to extract one or more molecular products from the liquid medium.

14. A method of forming a biomass reactor for producing a molecular product, comprising:
providing an enclosed fluid network including plural vertically offset receptacles, each receptacle containing a volume of a liquid medium and a microbial biomass growing in or on the liquid medium, each of the plural receptacles arranged in fluid communication to allow for a recirculating flow of the liquid medium to cascade through the plural receptacles such that the recirculating flow of the liquid medium flows into at least one first receptacle of the plural receptacles via at least one inlet and flows out of at least one final receptacle of the plural receptacles via at least one outlet;
providing a sump containing an additional volume liquid medium, the sump having a sump inlet in fluid communication with the at least one outlet of the enclosed fluid network, and a sump outlet; and
operating a pump to govern a flow of the liquid medium between the sump outlet and the inlet of the enclosed fluid network to establish and/or maintain the recirculating flow of the liquid medium.

15. A product formed by operating a biomass reactor according to claim 1.

16. A product according to claim 15, wherein the product comprises one or more of:
a. a syncytium product;
b. a bacterial protein product;
C. a product containing citrinin;
d. a product containing dihydro lysergic acid;
e. a product containing mevastatin;
f. a fermented product; and
g. a nucleic acid.

17. An installation for performing a method according to claim 13, the installation comprising a biomass reactor according to claim 1.

18. A biomass reactor according to claim 4, further comprising:
one or more sensors for sensing one or more parameters of the liquid medium flowing from the at least one final receptacle; and
means for making a correction to the one or more sensed parameters depending on the sensing.

19. A biomass reactor according to claim 18, wherein the plural receptacles are arranged as a vertically offset stack of receptacles.
